(19) United States
(12) Patent Application Publication
(10) Pub. No.: US 2025/0213211 A1
(43) Pub. Date: Jul. 3, 2025

(54) INTELLIGENT STETHOSCOPE, INTELLIGENT AUSCULTATION SYSTEM AND REMOTE AUSCULTATION SYSTEM

(71) Applicant: ScopeAround, Irvine, CA (US)

(72) Inventor: Yingjie Sun, Irvine, CA (US)

(21) Appl. No.: 18/406,378

(22) Filed: Jan. 8, 2024

(30) Foreign Application Priority Data
Dec. 27, 2023 (CN) 202323605812.2

(51) Int. Cl. A61B 7/04; A61B 7/02

(52) U.S. Cl. CPC A61B 7/04; A61B 7/026; A61B 2560/0247; A61B 2560/045; A61B 2560/0462; A61B 2562/0204

(57) ABSTRACT

Provided are an intelligent stethoscope, an intelligent auscultation system and a remote auscultation system. The intelligent stethoscope includes: a main control module, a sound processing module and a power supply module which are electrically connected to the main control module, and a stethoscope head provided with a main microphone electrically connected to the sound processing module. According to the present application, at least one secondary microphone is arranged at the stethoscope head, and collected sound is stored in a host or a patient side which is in communication connection with the host. The present application further provides the remote auscultation system. A patient and a doctor can perform visible remote auscultation through the system, so that the diagnosis quality can be improved.

### CROSS-REFERENCE TO RELATED APPLICATION

The present application claims priority to Chinese Patent Application No. 202323605812.2, filed on Dec. 27, 2023, the entire contents of which are incorporated herein by reference.

### TECHNICAL FIELD

The present disclosure relates to the technical field of medical devices, and in particular to an intelligent stethoscope, an intelligent auscultation system and a remote auscultation system.

### BACKGROUND

An acoustic stethoscope is the earliest stethoscope, and is also a medical diagnostic tool familiar to most people. An electronic stethoscope uses an electronic technology to amplify the body sound, thereby solving the problem of high noise of the acoustic stethoscope. The electronic stethoscope requires an electric signal of the sound wave of the converted sound for amplification and processing, thereby obtaining the best listening. Compared with the acoustic stethoscope, the stethoscopes are all based on the same physical principle.

The existing stethoscopes have a single function. For example, most electronic stethoscopes only have the functions of sound collection and playback. In a case that a patient has a sudden symptom and the cardiopulmonary sound at the onset of the disease cannot be saved in time, after the patient arrives at a medical institution, the symptom disappears, a doctor cannot know the information at the onset, which is not conducive to the diagnosis of the disease. In addition, many patients are inconvenient to move, cannot go to medical institutions for on-site auscultation at any time, and are prone to miss the best treatment opportunity, leading to the aggravation of the conditions of the patients.

### SUMMARY

An objective of the present disclosure is to provide a name, for solving at least one of the problems in the prior art.

To achieve the above objective, the present disclosure provides the following technical

### Solution

A first aspect of the present disclosure provides an intelligent stethoscope, including:
a host, including a circuit board and a circuit module arranged on the circuit board, the circuit module including a main control module, and a sound processing module and a power supply module which are electrically connected to the main control module; and a stethoscope head, provided with a main microphone, the main microphone being electrically connected to the sound processing module, where the sound processing module collects sound of an auscultation part through the main microphone and processes the collected sound.

In some embodiments, the intelligent stethoscope further includes a control module, where the control module is configured to control and manage a working state of the stethoscope, and includes several switches and several indicator lamps.

In some embodiments, the switches include at least one of a power switch, a mode switch, a communication switch, a volume switch and a recording switch.

In some embodiments, the intelligent stethoscope further includes at least one secondary microphone, where the secondary microphone is electrically connected to the sound processing module.

In some embodiments, the stethoscope head is provided with at least one secondary microphone; and
the sound processing module acquires ambient sound close to the main microphone through the at least one secondary microphone, and the ambient sound is used to eliminate ambient noise when the main microphone collects sound.

In some embodiments, the circuit module further includes a storage module configured to store the sound collected by the sound processing module.

In some embodiments, the intelligent stethoscope further includes an earphone interface and/or a loudspeaker; and
the circuit module further includes a power amplification module electrically connected to the main control module, the earphone interface and/or the loudspeaker.

A second aspect of the present disclosure provides an intelligent auscultation system, including:
the intelligent stethoscope and a patient side, where the intelligent stethoscope further includes a network communication module; the patient side is in communication connection with the intelligent stethoscope; and the patient side is equipped with an auscultation application program, acquires sound information collected by the intelligent stethoscope through the auscultation application program, and controls a working state of the intelligent stethoscope.

A third aspect of the present disclosure provides a remote auscultation system, including:
the intelligent auscultation system, a server and a diagnosis side, where the server is in communication connection with the patient side and the diagnosis side; and the intelligent stethoscope is in communication connection with the diagnosis side through the patient side and the server and transmits the sound collected by the intelligent stethoscope to the diagnosis side.

In some embodiments, each of the diagnosis side and the patient side includes a configuration for a network video call, thereby achieving remote visible auscultation.

Compared with the prior art, the present disclosure has the following advantages: according to the present application, at least one secondary microphone is configured to acquire the ambient noise during auscultation; the at least one secondary microphone is arranged at the stethoscope head so as to better collect the closer ambient noise when the main microphone collects the sound and provide the closest data support for the later noise reduction, thereby improving the sampling quality; in addition, the present application has many storage schemes, the collected sound can be stored in the host or the patient side which is in communication connection with the host, which is convenient for the doctor to know the disease history of the patient; and thirdly, the present application further provides the remote auscultation system, and the patient and the doctor can perform visible remote auscultation through the system, so that the diagnosis quality can be improved.

### BRIEF DESCRIPTION OF DRAWINGS

The drawings of the specification that constitutes a part of the present application are used to provide a further understanding of the present disclosure. The exemplary embodiments of the present disclosure and the description thereof are used to explain the present disclosure, and do not constitute an improper limitation of the present disclosure. In the drawings:

FIG. 1 is a schematic diagram of an overall structure of an intelligent stethoscope according to an embodiment of the present disclosure;

FIG. 2 is a structural schematic diagram of a stethoscope head according to an embodiment of the present disclosure;

FIG. 3 is a structural schematic diagram of a main control module according to an embodiment of the present disclosure;

FIG. 4 is a schematic diagram of a circuit structure of a sound processing module according to an embodiment of the present disclosure;

FIG. 5 is a schematic diagram of a circuit structure of a power amplification module according to an embodiment of the present disclosure;

FIG. 6 is a schematic diagram of a circuit structure of a power supply module according to an embodiment of the present disclosure; and

FIG. 7 is a schematic diagram of an overall structure of an intelligent auscultation system according to an embodiment of the present disclosure.

Description of reference numerals of the drawings: 1-intelligent stethoscope; 11-host; 1101-power switch; 1102-mode switch; 1103-communication switch; 1104-recording switch; 1105-indicator lamp; 1106-volume switch; 1107-earphone interface; 1108-loudspeaker; 12-stethoscope head; 1201-main microphone; 1202-secondary microphone; 13-wire; 2-mobile phone; 3-server; 4-diagnosis side.

### DETAILED DESCRIPTION OF THE EMBODIMENTS

The present disclosure will be described in detail below with reference to the accompanying drawings and in conjunction with embodiments. Various examples are provided by way of interpretation of the present disclosure and are not intended to limit the present disclosure. Indeed, it will be apparent to those skilled in the art that modifications and variations may be made in the present disclosure without departing from the scope or spirit of the present disclosure. For example, features shown or described as part of one embodiment may be used in another embodiment to produce yet another embodiment. Therefore, it is desirable that the present disclosure includes such modifications and variations falling within the scope of the appended claims and their equivalents.

In the description of the present disclosure, the terms “longitudinal”, “transverse”, “up”, “down”, “front”, “back”, “left”, “right”, “vertical”, “horizontal”, “top”, “bottom” and the like denote orientation or positional relationships based on those shown in the drawings and are intended for ease of description only and not to require that the present disclosure is necessarily constructed and operated in a particular orientation and therefore cannot be construed as limiting to the present disclosure. The terms “connection”, “connect” and “set” used in the present disclosure should be understood in a broad sense, for example, which may refer to a fixed connection or a detachable connection; which may refer to a direct connection or an indirect connection through intermediate components; which may refer to a wired electrical connection, a radio connection, or a wireless communication signal connection, and the specific meanings of the above terms may be understood by those of ordinary skill in the art according to a specific situation.

One or more examples of the present disclosure are shown in the accompanying drawings. The detailed description uses numeric and letter marks to refer to features in the drawings. Similar or like reference signs in the drawings and descriptions have been used to refer to similar or like parts of the present disclosure. As used herein, the terms “first”, “second” and “third” and the like are used interchangeably to distinguish one member from another and are not intended to denote the location or importance of individual members.

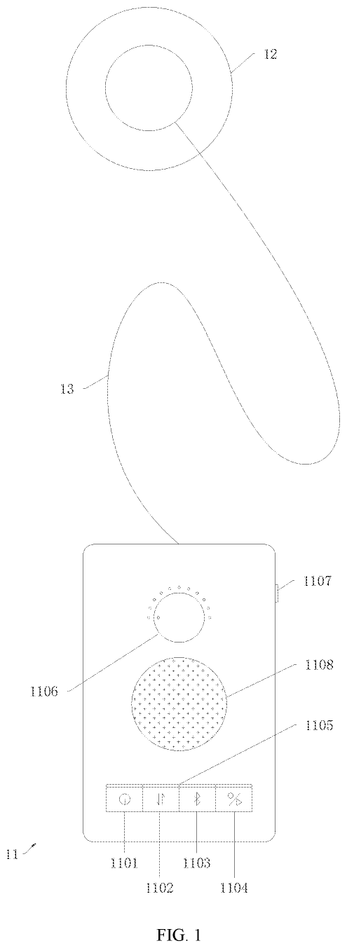

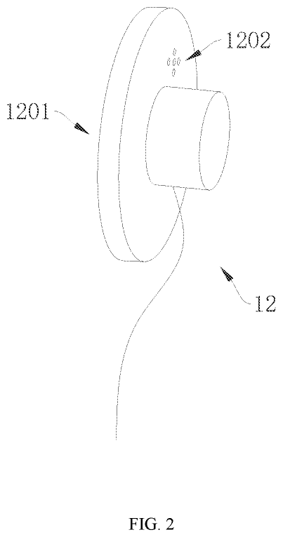

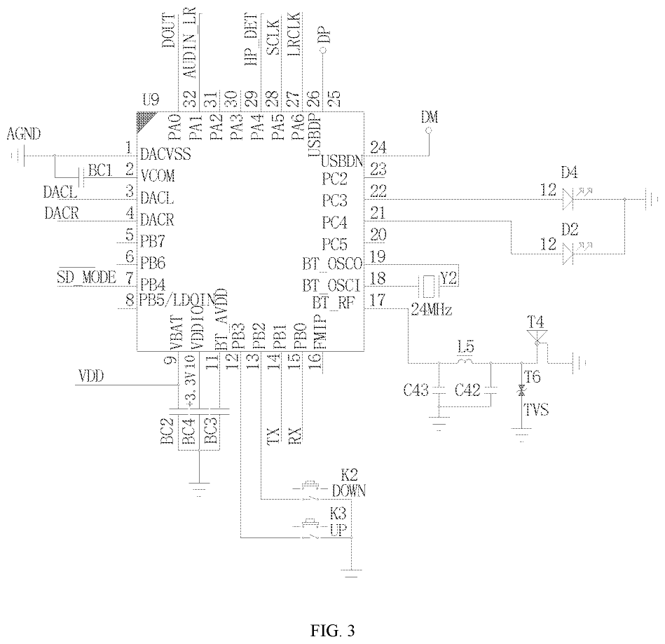

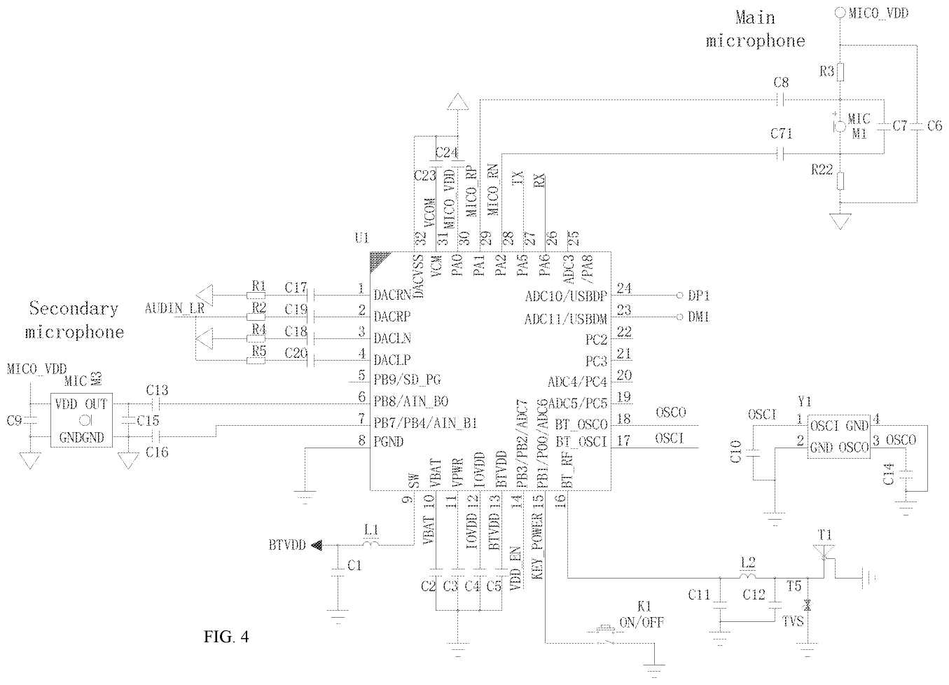

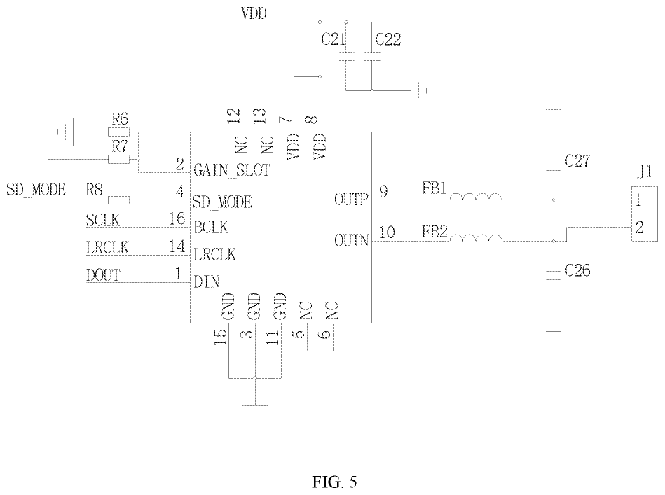

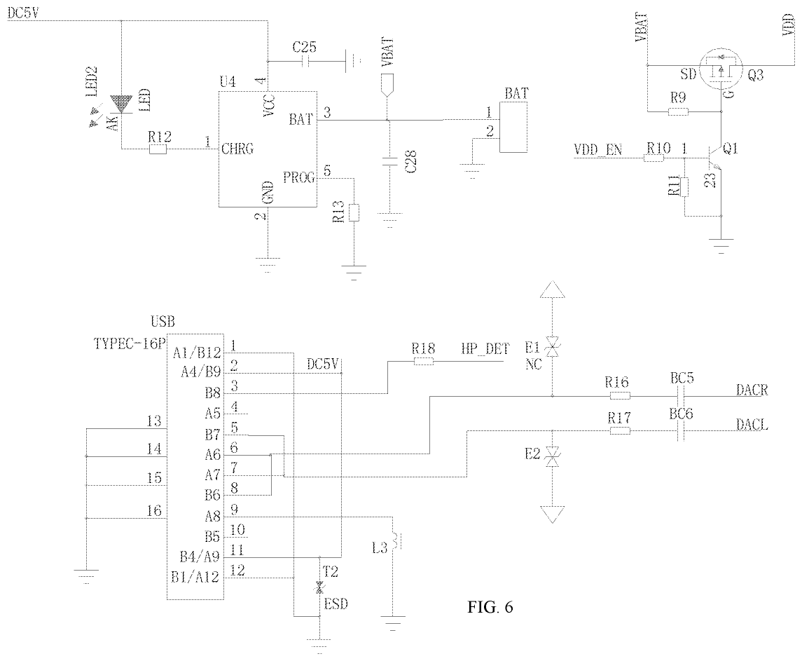

As shown in FIG. 1 to FIG. 6, according to embodiments of the present disclosure, an intelligent auscultation system is provided. The system includes an intelligent stethoscope 1; the intelligent stethoscope 1 includes a host 11 and a stethoscope head 12; the host 11 includes a shell, a circuit board arranged in the shell, and a circuit module arranged on the circuit board; and the circuit module includes a main control module, and a sound processing module, a network communication module, a control module and a power supply module which are electrically connected to the main control module. The stethoscope head 12 is provided with a main microphone 1201; the main microphone 1201 is electrically connected to the sound processing module through a wire 13; and the sound processing module collects sound of an auscultation part through the main microphone 1201 and processes the collected sound. The sound processing includes noise reduction and amplification.

The control module is configured to control and manage a state of the stethoscope, including startup and shutdown, network connection and the like. The control module includes a power switch 1101 for controlling startup and shutdown, and the power switch 1101 is arranged on the shell. An indicator lamp 1105 for displaying the on-off state is arranged on the shell.

In addition, the network connection refers to managing the connection state of the network communication module. For example, in this embodiment, the network communication module adopts a Bluetooth communication technology. The control module further includes a communication switch 1103, and manages the state of the network communication module through the communication switch 1103, including the on and off of the network, a pairing mode and the like. In this embodiment, the communication switch 1103 is also arranged on the shell of the host 11, an indicator lamp 1105 for the communication state is arranged on the shell, and the indicator lamp 1105 is configured to display the working state of Bluetooth. It is not excluded that the network module may further adopt a WIFI communication technology, a stroboscopic communication technology and the like.

Due to the different features of heart sound and pulmonary sound, according to the present application, during auscultation, it is necessary to select different modes and adjust the volume according to the auscultation part. To achieve the above function, the control module further includes a volume switch 1106 for adjusting the volume of the sound, a mode switch 1102 for switching the heart sound or the pulmonary sound, and the like. The volume switch 1106 adopts a knob adjusting switch. The volume switch 1106 and the mode switch 1102 are arranged on the shell of the host 11.

To further remove the ambient noise, the present application is further provided with at least one secondary microphone 1202. In one of the embodiments, one of the secondary microphones 1202 is arranged at the stethoscope head 12, is configured to acquire the ambient sound close to the main microphone 1201, and is favorable for reducing the quality of noise reduction. In addition, the secondary microphone may be arranged on the host.

In some cases, during auscultation, it is necessary to save audio information of the auscultation for the later playback as a reference when doctors diagnose the disease. For example, when the patient has a sudden symptom in some cases, but the symptom disappears when the patient is sent to the medical institution, the doctor cannot master the information of the cardiopulmonary sound of the patient at the time of onset. Therefore, the present application is provided with a storage function. Specifically, the circuit module in the present application further includes a storage module, and the control module includes a recording switch 1104. In this embodiment, the recording switch 1104 is also arranged on the shell; furthermore, an indicator lamp 1105 for displaying the recording state is also arranged on the shell. During auscultation, the recording switch 1104 is pressed, and the recording module will record the audio information of a cardiopulmonary signal during auscultation. Of course, it may be set as follows: when the sound processing module collects sound at each time, the storage module will store the collected sound one by one and manage stored sound files according to a time sequence. When the storage space is insufficient, the sound files with earlier dates can be deleted or covered to realize cyclic recording.

According to the present application, the cardiopulmonary sound can be listened through an earphone or loudspeaker 1108 during auscultation or when the recorded audio is played. Therefore, the shell is provided with an earphone interface 1107 and/or a loudspeaker 1108, the earphone interface 1107 and/or the loudspeaker 1108 are/is electrically connected to the power amplification module respectively, and the power amplification module is electrically connected to the main control module. During playback, the volume of the sound can be adjusted through the volume switch 1106. Of course, other audio playing devices may also be connected through the network module.

More specifically, in the above scheme, an AC7006 chip may serve as a chip of the sound processing module which is responsible for collecting the cardiopulmonary sound. An AC6966 chip serves as a chip of the main control module; furthermore, the chip has Bluetooth communication ability and can establish a communication connection with a Bluetooth device.

The host 11 and the stethoscope head 12 of the stethoscope 1 may also adopt an integrated design, and the stethoscope head 12 may be mounted at one end of the host 11. In addition, in some embodiments, the stethoscope head is provided with a communications module and a battery module electrically connected to the communications module; and the main microphone 1201 and the secondary microphone 1202 are electrically connected to the communications module, and are in wireless communication connection with the circuit module of the host through the communication module.

The network communication module of the present application is configured to connect a mobile phone 2; the mobile phone 2 is provided with a stethoscope APP; and the state of the intelligent stethoscope 1 can be controlled through the stethoscope APP, for example, switching the mode of the heart sound and the pulmonary sound, recording the audio information, playing the collected cardiopulmonary sound or the recorded cardiopulmonary sound, and adjusting the volume.

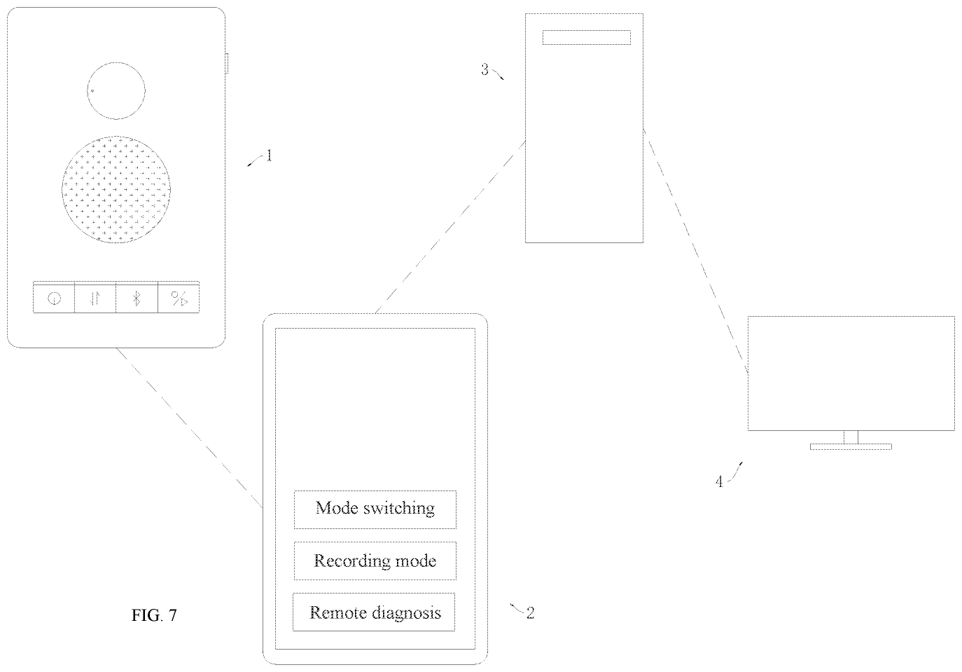

Referring to FIG. 7, in some embodiments, the present application can realize remote auscultation through the mobile phone 2 equipped with the stethoscope APP. Specifically, the present application further includes a server 3 and a diagnosis side 4, and the diagnosis side 4 and the mobile phone 2 are in communication connection with the server 3. The diagnosis side 4 is used by the doctor, and the doctor can acquire the auscultation information of the patient through the diagnosis side 4. Furthermore, the diagnosis side 4 is further provided with a microphone, a camera and a display screen. The patient can make video and voice calls with the doctor through the mobile phone 2, and the doctor can observe the state of the patient and make further diagnosis in combination with the cardiopulmonary sound of the patient, thereby improving the diagnosis quality. The diagnosis side 4 may be a mobile phone 2, a tablet computer or a PC computer.

In addition, the diagnosis side 4 can control the intelligent stethoscope 1 of the patient according to requirements, for example, adjusting the volume.

A remote diagnosis system of the present application includes an intelligent stethoscope 1, a patient side, a server 3 and a diagnosis side 4. The patient side includes but not limited to the mobile phone 2 or other electronic devices capable of performing a video call and network communication. During remote diagnosis, the intelligent stethoscope 1 is connected to the server 3 through the patient side, and then establishes a communication connection with the diagnosis side 4 of the server 3. The patient can transmit the cardiopulmonary sound collected by the intelligent stethoscope 1 to the diagnosis side 4, and the patient can also perform an online video call with the doctor through the patient side and the diagnosis side 4 to realize visible remote auscultation.

The foregoing is merely a preferred embodiment of the present disclosure and is not intended to limit the present disclosure which may be subject to various modifications and variations to those skilled in the art. Any modification, equivalent replacement, improvement, etc. made within the spirit and principles of the present disclosure should be included in the scope of protection of the present disclosure.

What is claimed is:

1. An intelligent stethoscope,
comprising:
a host, comprising a circuit board and a circuit module arranged on the circuit board, the circuit module comprising a main control module, and a sound processing module and a power supply module which are electrically connected to the main control module; and
a stethoscope head, provided with a main microphone, the main microphone being electrically connected to the sound processing module,
wherein the sound processing module collects sound of an auscultation part through the main microphone and processes the collected sound.

2. The intelligent stethoscope according to claim 1,
further comprising a control module, wherein the control module is configured to control and manage a working state of the stethoscope, and comprises several switches and several indicator lamps.

3. The intelligent stethoscope according to claim 2,
wherein the switches comprise at least one of a power switch, a mode switch, a communication switch, a recording switch and a volume switch.

4. The intelligent stethoscope according to claim 1,
further comprising at least one secondary microphone, wherein the secondary microphone is electrically connected to the sound processing module.

5. The intelligent stethoscope according to claim 4,
wherein the stethoscope head is provided with at least one secondary microphone; and
the sound processing module acquires ambient sound close to the main microphone through the at least one secondary microphone, and the ambient sound is used to eliminate ambient noise when the main microphone collects sound.

6. The intelligent stethoscope according to claim 1,
wherein the circuit module further comprises a storage module configured to store the sound collected by the sound processing module.

7. The intelligent stethoscope according to claim 1,
further comprising an earphone interface and/or a loudspeaker,
wherein the circuit module further comprises a power amplification module electrically connected to the main control module, the earphone interface and/or the loudspeaker.

8. An intelligent auscultation system,
comprising an intelligent stethoscope and a patient side, wherein the intelligent stethoscope comprises: a host, comprising a circuit board and a circuit module arranged on the circuit board, the circuit module comprising a main control module, and a sound processing module and a power supply module which are electrically connected to the main control module; and a stethoscope head, provided with a main microphone, the main microphone being electrically connected to the sound processing module, wherein the sound processing module collects sound of an auscultation part through the main microphone and processes the collected sound; and the intelligent stethoscope further comprises a network communication module, and the patient side is in communication connection with the intelligent stethoscope,
wherein the patient side is equipped with an auscultation application program, acquires sound information collected by the intelligent stethoscope through the auscultation application program, and controls a working state of the intelligent stethoscope.

9. A remote auscultation system,
comprising:
an intelligent stethoscope and a patient side, wherein the intelligent stethoscope comprises: a host, comprising a circuit board and a circuit module arranged on the circuit board, the circuit module comprising a main control module, and a sound processing module and a power supply module which are electrically connected to the main control module; and a stethoscope head, provided with a main microphone, the main microphone being electrically connected to the sound processing module, wherein the sound processing module collects sound of an auscultation part through the main microphone and processes the collected sound; and the intelligent stethoscope further comprises a network communication module, and the patient side is in communication connection with the intelligent stethoscope, wherein the patient side is equipped with an auscultation application program, acquires sound information collected by the intelligent stethoscope through the auscultation application program, and controls a working state of the intelligent stethoscope; and
a server and a diagnosis side, wherein the server is in communication connection with the patient side and the diagnosis side, and the intelligent stethoscope is in communication connection with the diagnosis side through the patient side and the server, and transmits the sound collected by the intelligent stethoscope to the diagnosis side.

10. The remote auscultation system according to claim 9,
wherein each of the diagnosis side and the patient side comprises a configuration for a network video call, thereby achieving remote visible auscultation.
